(19) United States
(12) Patent Application Publication
(10) Pub. No.: US 2012/0157922 A1
(43) Pub. Date: Jun. 21, 2012

(54) INSERTER MECHANISMS

(75) Inventors: Avraham Shekalim, Nesher (IL); Noam Peleg, Gan-Ner (IL); Gilad Ben-Tzur, Gan-Ner (IL)

(73) Assignee: MEDX-SET LTD., Tirat-Carmel (IL)

(21) Appl. No.: 13/388,999

(22) PCT Filed: Aug. 5, 2010
(86) PCT No.: PCT/IB2010/053548

§ 371 Date: Mar. 7, 2012

Related U.S. Application Data
(60) Provisional application No. 61/231,740, filed on Aug. 6, 2009.
(60) Provisional application No. 61/290,534, filed on Dec. 29, 2009.

(51) Int. Cl. A61B 17/34

(52) U.S. Cl. 604/164.12; 604/164.01

(57) ABSTRACT

An inserter for inserting a needle of a drug delivery device through the skin of a user, the inserter having an inserter housing (30); a base section (60) associated with the inserter housing such that an interaction between the inserter housing and the base allows the inserter housing to tilt in relation to the base; a manually displaceable plunger (20) deployed in the housing, the plunger displaceable between a stand-by position and an insertion position; and an insertion needle (6) extending from the plunger in an insertion direction; wherein the manual displacement of the plunger is achieved by applying a force greater than a required force threshold so as to free the plunger from the stand-by position.

### FIELD OF THE INVENTION

The present invention relates to inserter mechanisms for insertion of a drug delivery device through the skin of a user, and most preferably, for insertion of a flexible cannula which is then left in place for a period of time for continuous or intermittent delivery of a drug into the body. It will be appreciated that while the inserter mechanisms of the present invention may be used in combination with each other to provide a unique insertion device, each of the mechanisms may be used individually to benefit in combination with known insertion mechanisms. More specifically, the present invention relates to a manually operated insertion mechanism and an insertion device configured to allow for a user selected insertion angle within the range of tilt motion provided by the insertion device.

It is known in the art to provide insertion devices that insert the cannula at an angle other than 90° to the surface of the patient's skin. This is generally accomplished by use of an inserter having a single pre-determined angle. Alternatively, the inserter may be able to rotate between a number of pre-determined angular orientations. None of the current devices provide for orientation of the cannula at substantially any user selected angle within the range of tilt motion provided by the insertion device.

It is also known to provide high speed insertion of the cannula by means of power driven, usually spring power, mechanisms. Manual insertion of a cannula is generally a slow process accomplished using a syringe type inserter. None of the current devices provide for manual high speed insertion of the cannula.

There is, therefore, a need for an insertion device that provides for manual high speed insertion of the cannula at substantially any user selected insertion angle within the range of tilt motion provided by the insertion device.

### SUMMARY OF THE INVENTION

The present invention is an insertion device that provides for manual high speed insertion of the cannula at substantially any user selected insertion angle within the range of tilt motion provided by the insertion device.

According to the teachings of the present invention there is provided, an inserter for inserting a flexible cannula of a drug delivery device through the skin of a user, the inserter comprising: (a) an inserter housing; (b) a base section detachably connected to the inserter housing such that an interaction between the inserter housing and the base allows the inserter housing to tilt in relation to the base; (c) a manually displaceable plunger deployed in the housing, the plunger displaceable between a stand-by position and an insertion position, the plunger being held in the stand-by position by retaining elements configured in at least one of the inserter housing and the plunger; (d) an insertion needle extending from the plunger in an insertion direction; and (e) a flexible cannula is deployed on the insertion needle for insertion; wherein the manual displacement of the plunger is achieved by applying a force greater than a required force threshold so as to free the plunger from the stand-by position.

There is also provided according to the teachings of the present invention, an inserter for inserting a cannula of a drug delivery device through the skin of a user, the inserter comprising: (a) an inserter housing; (b) a manually displaceable plunger deployed in the housing, the plunger displaceable between a stand-by position and an insertion position, the plunger being held in the stand-by position by retaining elements configured in at least one of the inserter housing and the plunger; (c) an insertion needle extending from the plunger is an insertion direction; and (d) a flexible cannula is deployed on the insertion needle for insertion; wherein the manual displacement of the plunger is achieved by applying a force greater than a required force threshold so as to free the plunger from the stand-by position.

According to a further teaching of the present invention, the plunger is configured with at least two retaining spring elements that releasably engage the inserter housing so as to hold the plunger in the stand-by position.

According to a further teaching of the present invention, the retaining spring elements are outwardly biased such that when the force greater than the required force threshold is applied, the retaining spring elements are inwardly displaced.

According to a further teaching of the present invention, the inserter housing is configured with at least two retaining spring elements that releasably engage the plunger so as to hold the plunger in the stand-by position.

According to a further teaching of the present invention, the retaining spring elements are inwardly biased such that when the force greater than the required force threshold is applied, the retaining spring elements are outwardly displaced.

According to a further teaching of the present invention, there is also provided an automatic retraction mechanism configured to automatically retract the insertion needle.

According to a further teaching of the present invention, the inserter housing is detachably connected to the base.

There is also provided according to the teachings of the present invention, an inserter for inserting a cannula of a drug delivery device through the skin of a user, the inserter comprising: (a) an inserter housing; (b) a base section releasably connected to the inserter housing; (c) an insertion needle extending from the plunger is an insertion direction; and (d) a flexible cannula is deployed on the insertion needle for insertion; wherein at least one of the inserter housing and the base includes at least one component that allow the inserter housing to tilt in relation to the base.

According to a further teaching of the present invention, the base section includes a cannula trap configured to allow the inserter housing to tilt in relation to the base section.

According to a further teaching of the present invention, the inserter housing is releasably connected to the cannula trap.

According to a further teaching of the present invention, the cannula trap tilts by rotating about an axis that is parallel to a bottom surface of the base section and passes through the base section.

According to a further teaching of the present invention, at least one of the inserter housing and the base includes elements that bias the cannula trap to an upright position, such that upon completion of an insertion process with the cannula trap in a tilt position, the cannula trap is automatically returned to an upright position.

According to a further teaching of the present invention, the inserter housing is configured with a cut-out region that accommodates tilting of the inserter housing in relation to the base.

According to a further teaching of the present invention, at least one of the inserter housing and the base includes elements that bias the inserter housing to an upright position, such that upon completion of an insertion process with the inserter housing in a tilt position, the inserter housing is automatically returned to an upright position.

### BRIEF DESCRIPTION OF THE DRAWINGS

The invention is herein described, by way of example only, with reference to the accompanying drawings, wherein:

FIG. 1A is a cross sectional view of a first preferred embodiment of an insertion device constructed and operational according to the teachings of the present invention, shown here in an upright position and in a stand-by deployment;

FIG. 1B is a cross sectional view of the insertion device of FIG. 1A, shown in an upright position and in a insertion deployment;

FIG. 1C is a cross sectional view of the insertion device of FIG. 1A, shown in an upright position and in a retracted deployment;

FIG. 1D is a detail of FIG. 1A;

FIG. 1E and a detail of the plunger element of the inserter device of FIG. 1A;

FIG. 2A is an isometric top view of a base section constructed and operational according to the teachings of the present invention;

FIG. 2B is a cross sectional view of the base section of FIG. 2A shown with the cannula trap in the upright position;

FIG. 2C is a cross sectional view of the base section of FIG. 2A shown with the cannula trap in a tilted position;

FIG. 2D is a detail of the cannula trap of the base section of FIG. 2A;

FIG. 3A is a cross sectional view of the insertion device of FIG. 1A shown here in an tilted position and in a stand-by deployment;

FIG. 3B is a cross sectional view of the insertion device of FIG. 1A, shown in an tilted position and in a insertion deployment;

FIG. 3C is a cross sectional view of the insertion device of FIG. 1A, shown in an tilted position and in a retracted deployment;

FIG. 4A is an isometric view of the insertion device of FIG. 1A shown here in an upright position and in a stand-by deployment;

FIG. 4B is an isometric view of the insertion device of FIG. 1A, shown in an upright position and in an insertion deployment;

FIG. 5A is an isometric view of the insertion device of FIG. 1A shown here in an tilted position and in a stand-by deployment;

FIG. 5B is an isometric view of the insertion device of FIG. 1A, shown in a tilted position and in an insertion deployment;

FIG. 6A is an isometric view of the inserter device of FIG. shown with a protective cap constructed and operational according to the teachings of the present invention;

FIG. 6B is a side elevation of the device of FIG. 6A;

FIG. 6C is a cross sectional view of the device of FIG. 6A;

FIG. 7A is a side elevation of the inserter device of FIG. 1A shown here with a plunger locking-element in the locked position;

FIG. 7B is a side elevation of the inserter device of FIG. 1A shown here with a plunger locking-element partially removed;

FIGS. 7C and 7D are isometric views of FIG. 7A;

FIG. 7E is a cross sectional view of FIG. 7A; and

FIG. 8 is a cross sectional view of a second preferred embodiment of an inserter device constructed and operational according to the teachings of the present invention, shown here in a stand-by deployment.

### DESCRIPTION OF THE PREFERRED EMBODIMENTS

The present invention is an insertion device that provides for manual high speed insertion of the cannula at substantially any user selected insertion angle within the range of tilt motion provided by the insertion device.

The principles and operation of an insertion device according to the present invention may be better understood with reference to the drawings and the accompanying description.

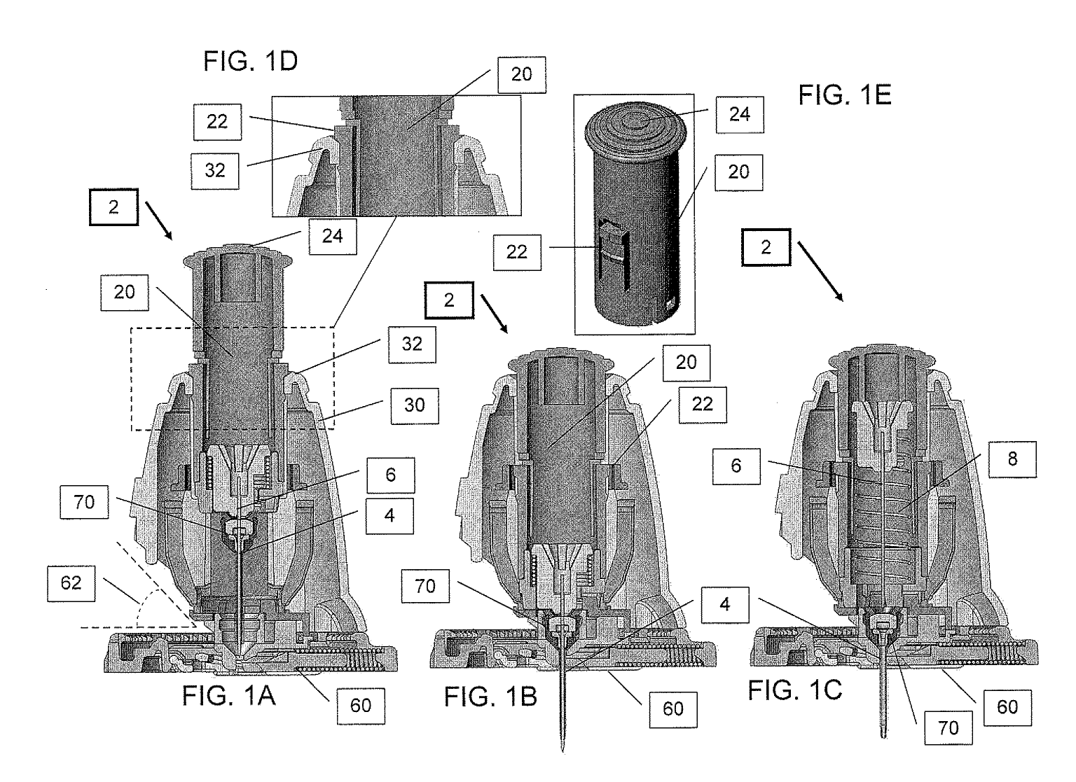

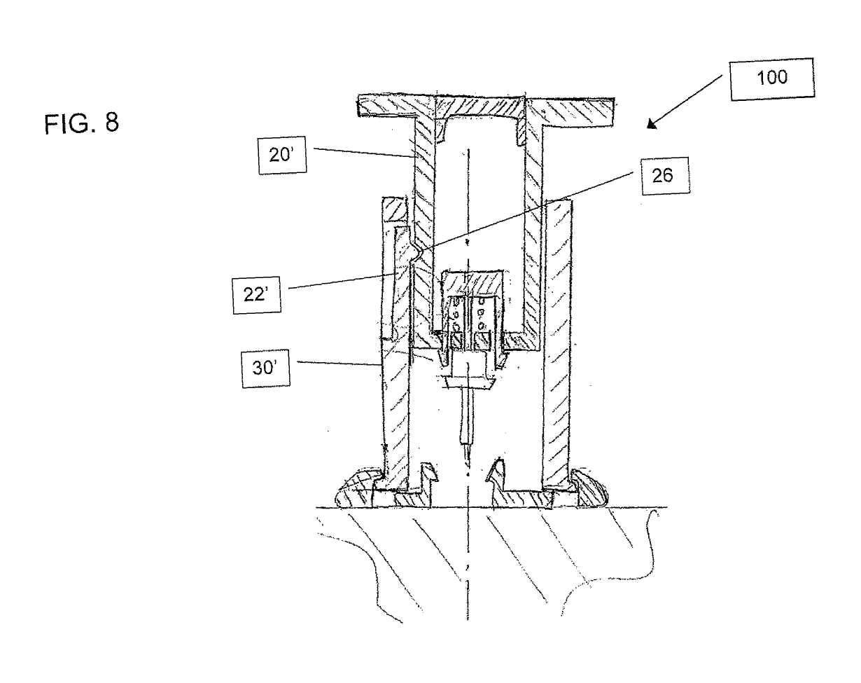

By way of introduction, illustrated herein are a number of features that may be used to benefit either individually or in combination. An illustrative example of a first preferred tiltable embodiment of the inserter according to some aspects of the present invention is shown in FIGS. 1-7 of the attached drawings. A non-tiltable second preferred embodiment of the inserter according to other aspects of the present invention is shown in FIG. 8 of the attached drawings. It will be understood that those elements that are common to more than one drawing figure are identified with the same reference numeral.

It is a feature of the inserters of the present invention that they are configured so as to be manually operated for insertion of the cannula 4 while retraction of the carrier needle 6 may be automatically retracted by use of a spring 8.

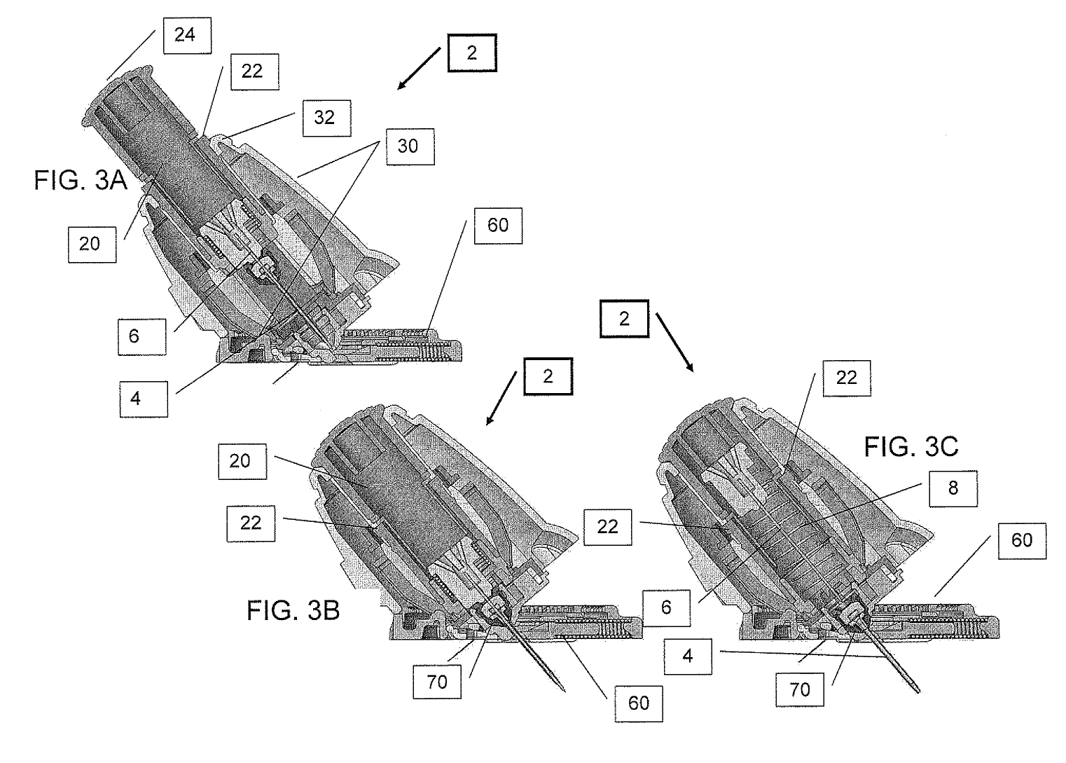

To this end, as illustrated in FIGS. 1-7, the insertion plunger 20 is deployed in the inserter housing 30 and retained in a stand-by deployment (FIGS. 1A and 3A) such that when a force that exceeds a required threshold is applied by a user, such as by the user's thumb for example, to the insertion plunger 20 in an insertion direction, the insertion plunger 20 breaks free of the stand-by deployment and moves forward so as to insert the carrier needle 6 and cannula 4 into the desired tissue (FIGS. 1B and 3B). The stand-by deployment may be maintained by a releasable interlocking arrangement configured to require application of at least a threshold value of force to release it, as illustrated in the drawings here. Alternatively, although not illustrated, the stand-by deployment may be maintained by a frangible (breakable) connecting element which would define the threshold force and also provide tamper evidence.

It should be noted that due to the amount of force required to pass the force threshold and the short distance traveled by the insertion plunger 20, it is virtually impossible to noticeably affect insertion before initial penetration sufficient to achieve painless insertion (breaking of nerve cells). There is, however, a possibility that the velocity profile may drop off slightly for the remaining part of the insertion motion, but this is less critical since it is painless.

As illustrated in the drawings, the first preferred tiltable embodiment 2 of the inserter mechanism of the present invention includes an inserter housing 30 that is configured with a retaining-lip portion 32 and an insertion plunger 20. The insertion plunger 20 is configured with at least two retaining spring elements 22 that releasably engage the retaining-lip 32 when the insertion plunger 20 is deployed in the stand-by deployment (FIGS. 1A and 3A). The insertion plunger 20 also includes a cap 24 to which force is manually applied in order to operate the insertion process. It should be noted that the number of retaining spring elements may be varied dependent on the actual design and may range from as few as one to as many as desired or required for the particular application.

Extending from the insertion plunger 20 in a direction toward the tissue into which the cannula is to be inserted is a cannula 4 deployed on a carrier needle 6.

In operation, this first preferred embodiment 2 of the insertion device of the present invention is placed against the skin surface of the patient with the base section 60 being removably attached to the skin. Force is manually applied to cap 24 and once the required force threshold is reached, the retaining spring elements 22 move inward releasing the insertion plunger 20 from the stand-by deployment and allowing it to move toward the surface of the tissue.

Upon completion of the outward insertion stroke of the insertion needle 6, the cannula 4 is forced into the cannula trap 64 and the retraction spring 8 is released so as to retract the insertion needle 6 leaving the cannula 4 in place, now attached to the base section 60 (FIGS. 1C and 3C). The inserter housing 30 is then disconnected from the base section 60 which is left attached to the patient with the cannula inserted into the target tissue.

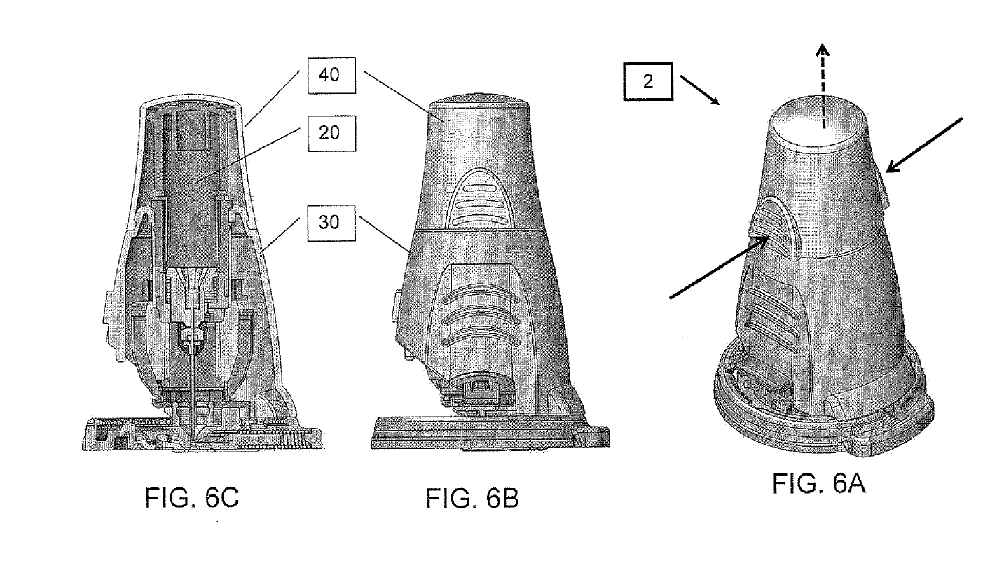

FIGS. 5A-6E illustrate a protective cap 40 (FIGS. 5A-5C) and a plunger locking-element 50 (FIGS. 6A-6E). The protective cap 40 and plunger locking-element 50 are designed to protect from unintentional operation of the insertion plunger 20 during shipping and storage up until the time of use.

The protective cap 40 is deployed on the inserter housing 30 so as to enclose the portion of the insertion plunger 20 that extends outside of the inserter housing 30.

The plunger locking-element 50 is deployed so as to extend along a portion of the inserter housing 30 and lockingly engage the insertion plunger 20 so as to prevent movement of the insertion plunger.

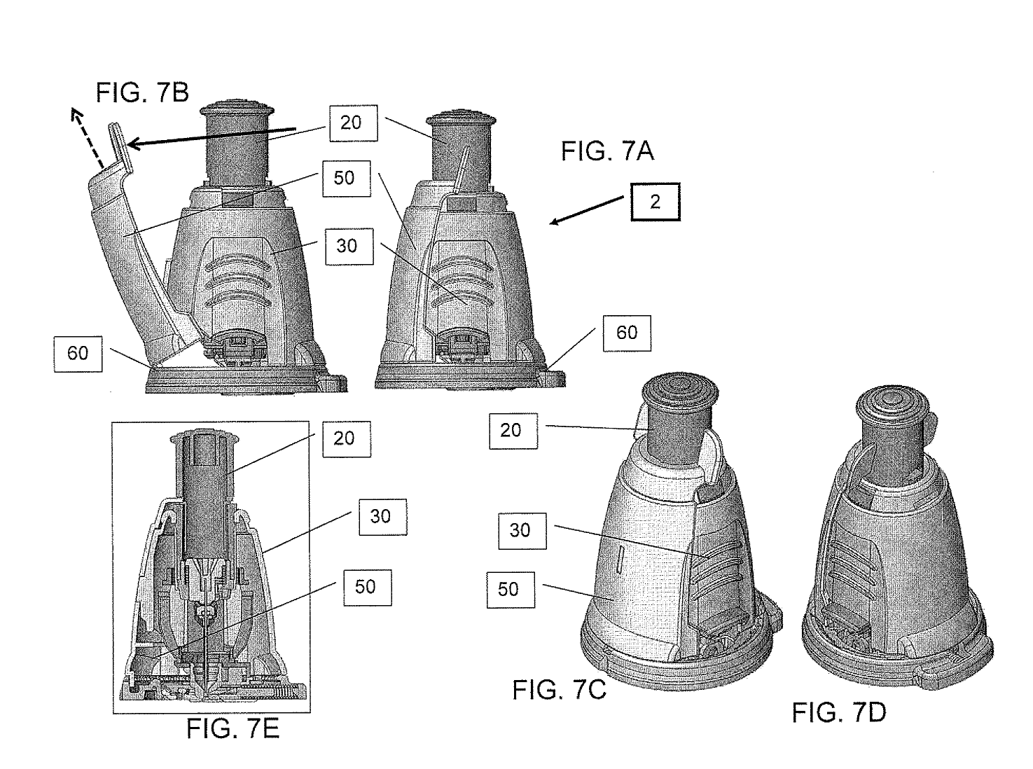

FIG. 7 illustrates a second preferred embodiment of the insertion mechanism of the present invention. This embodiment 100 of the insertion mechanism of the present invention includes an inserter housing 30′ that is configured with at least two retaining spring elements 22′ and an insertion plunger 20′. The insertion plunger 20′ is configured with corresponding retaining indentations 26 that are releasably engaged by the retaining spring elements 22′ when the insertion plunger 20′ is deployed in the stand-by deployment. The insertion plunger 20′ also includes a cap to which force is manually applied in order to operate the insertion process.

Operation of this second preferred embodiment 100 of the insertion device of the present invention is similar to that of the embodiment described above. However, when force is manually applied to the insertion plunger cap and the required force threshold is reached, the retaining spring elements 22′ move outwardly releasing the insertion plunger 20′ from the stand-by deployment and allowing it to move toward the surface of the tissue.

While the embodiment illustrated in FIG. 8 is shown in a non-tiltable configuration, it will be appreciated that this embodiment 100 may also be implemented with a base section 60′ that allows the inserter housing 30′ to be tilted in relation to the base. This is also true of the embodiment 2 of FIGS. 1-7 which is illustrated in a tiltable configuration but can also be provided in a non-tiltable configuration

As mentioned above, once the cannula has been inserted, the inserter housing 30′ is disconnected from the base section 60′ which is left attached to the patient.

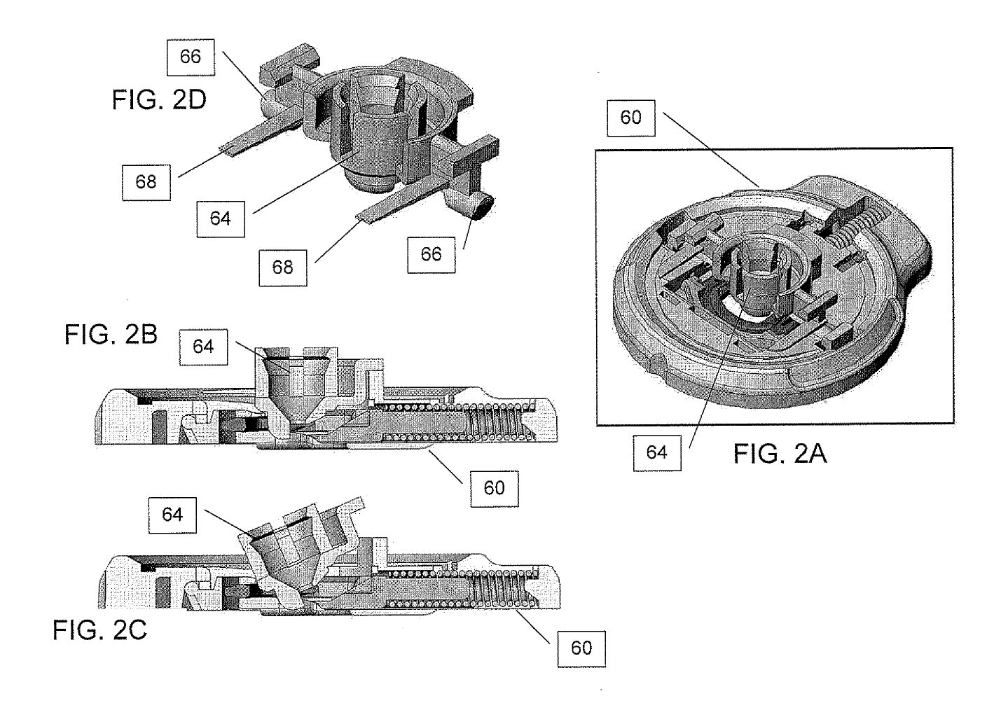

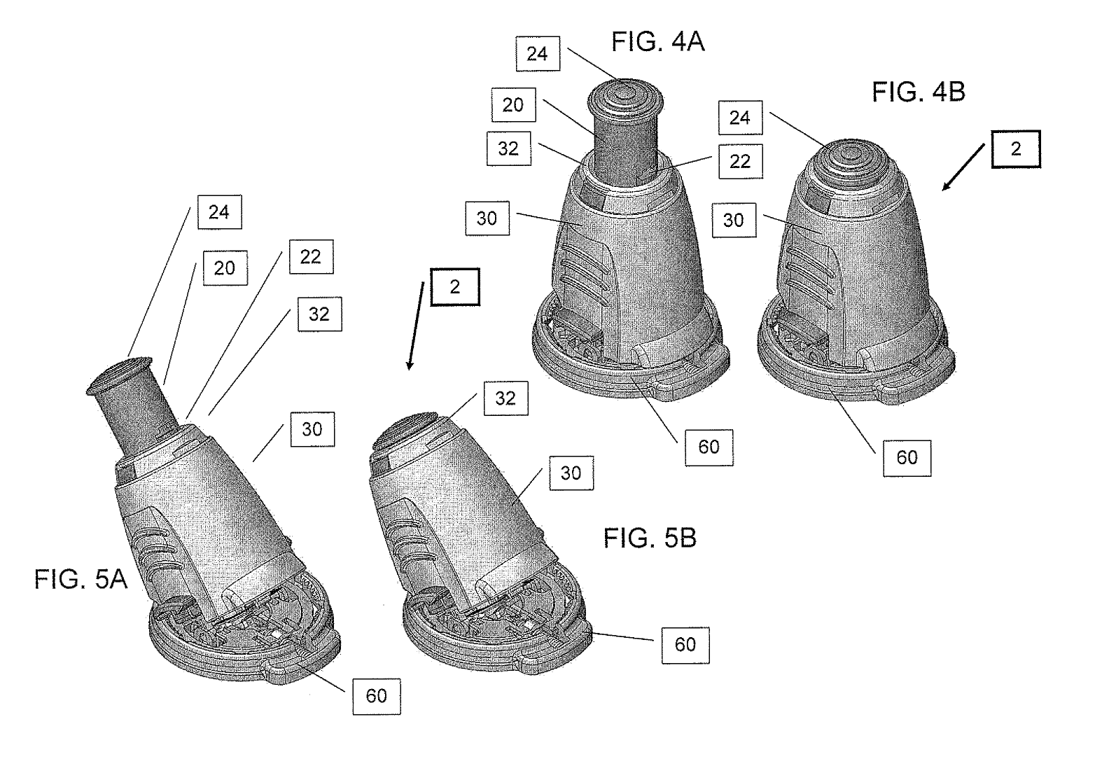

It is another feature of the inserters of the present invention is that they are configured so as to be tiltable. Therefore, it will be readily appreciated from FIGS. 1 and 3-7, that the insertion mechanism of embodiment 2 of the present invention may be operated in either an upright position (FIGS. 1 and 4) that is substantially perpendicular to the surface of the tissue or in a tilted position (FIGS. 3 and 5). An example of a suitable arrangement for providing the ability to tilt the inserter housing in relation to the base section is illustrated in FIGS. 2A-2D. However, it will be appreciated that substantially any suitable base section to housing interconnection may be used.

As illustrated in FIG. 1A, the inserter housing 30 has a “cut-out” region 62 that accommodates tilting of the inserter housing 30 in relation to the disconnectable base section 60 which remains attached to skin of the patient after the cannula 4 has been inserted.

The tilting is made possible by the interaction of the cannula trap 64 and the base section 60 to which it is attached. As illustrated in FIG. 2D, the cannula trap 64 element includes pivot components 66 which allow the cannula trap 64 to rotate about an axis that is substantially parallel to the plane of the bottom surface of the base 60, and thereby substantially parallel to the surface of the skin to which the base is attached. The inserter housing 30 which includes the actual insertion mechanism is interconnected to the base section 60 by the cannula trap 64, thereby allowing the inserter housing 30 to rotate (tilt) with the cannula trap 64.

As seen in FIGS. 3A-3C, in this illustrative embodiment the line of insertion along which the insertion needle 6 and cannula 4 travel intersects the axis of rotation of the cannula trap 64.

The return leaf springs 68 serve to keep the cannula trap 64 and the inserter housing 30 in a normally upright position and return the cannula trap 64 and the inserter housing 30 to such a position after the insertion process has been completed in order to facilitate disconnection of the inserter housing 30 from the base 60.

In operation, the tilt feature of the insertion device of the present invention is placed against the skin surface of the patient with the inserter housing 30 connected to the base 60, the base section which is removably attached to the skin. The inserter housing 30 is tilted to the desired angle and force is applied to the plunger cap 24 until the required force threshold is reached which releases the plunger 20 and drives the insertion needle 6 with the cannula 4 attached into the patient.

Upon completion of the outward stroke of the insertion needle, the cannula 4 is forced into the cannula trap 64 and the retraction spring 8 retracts the insertion needle 6 leaving the cannula 4 in place, now attached to the base section and inserted into the tissue of the patient. The inserter housing 30 and the cannula trap 64 are brought back to an upright position and the inserter housing 30 is disconnected from the base section 60 which is left attached to the patient.

It should be noted that while the cannula is inserted into the patient at an angular orientation, for purposes of connection to the infusion apparatus the top section 70 of the cannula 4 that engages the cannula trap 64 needs to be in an upright orientation after the insertion process is completed. To that end, when the return leaf springs 68 return the cannula trap 64 to the normally upright position after the insertion process has been completed the top section 70 of the cannula 4 is brought to an upright orientation while the portion of the cannula that remains in the patient remains at the angular orientation.

It will be appreciated that the above descriptions are intended only to serve as examples and that many other embodiments are possible within the scope of the present invention as defined in the appended claims.

What is claimed is:

1. An inserter for inserting a flexible cannula of a drug delivery device through the skin of a user, the inserter comprising:
(a) an inserter housing;
(b) a base section detachably connected to said inserter housing such that an interaction between said inserter housing and said base allows said inserter housing to tilt in relation to said base;
(c) a manually displaceable plunger deployed in said housing, said plunger displaceable between a stand-by position and an insertion position, said plunger being held in said stand-by position by retaining elements configured in at least one of said inserter housing and said plunger;
(d) an insertion needle extending from said plunger in an insertion direction; and
(e) a flexible cannula is deployed on said insertion needle for insertion;
wherein said manual displacement of said plunger is achieved by applying a force greater than a required force threshold so as to free said plunger from said stand-by position.

2. An inserter for inserting a cannula of a drug delivery device through the skin of a user, the inserter comprising:
(a) an inserter housing;
(b) a manually displaceable plunger deployed in said housing, said plunger displaceable between a stand-by position and an insertion position, said plunger being held in said stand-by position by retaining elements configured in at least one of said inserter housing and said plunger;
(c) an insertion needle extending from said plunger is an insertion direction; and
(d) a flexible cannula is deployed on said insertion needle for insertion;
wherein said manual displacement of said plunger is achieved by applying a force greater than a required force threshold so as to free said plunger from said stand-by position.

3. The inserter of claim 2, wherein said plunger is configured with at least two retaining spring elements that releasably engage said inserter housing so as to hold said plunger in said stand-by position.

4. The inserter of claim 3, wherein said retaining spring elements are outwardly biased such that when said force greater than said required force threshold is applied, said retaining spring elements are inwardly displaced.

5. The inserter of claim 2, wherein said inserter housing is configured with at least two retaining spring elements that releasably engage said plunger so as to hold said plunger in said stand-by position.

6. The inserter of claim 5, wherein said retaining spring elements are inwardly biased such that when said force greater than said required force threshold is applied, said retaining spring elements are outwardly displaced.

7. The inserter of claim 2, further including an automatic retraction mechanism configured to automatically retract said insertion needle.

8. The inserter of claim 2, wherein said inserter housing is detachably connected to said base.

9. An inserter for inserting a cannula of a drug delivery device through the skin of a user, the inserter comprising:
(a) an inserter housing;
(b) a base section releasably connected to said inserter housing;
(c) an insertion needle extending from said plunger is an insertion direction; and
(d) a flexible cannula is deployed on said insertion needle for insertion;
wherein at least one of said inserter housing and said base includes at least one component that allow said inserter housing to tilt in relation to said base.

10. The inserter of claim 9, wherein said base section includes a cannula trap configured to allow said inserter housing to tilt in relation to said base section.

11. The inserter of claim 10, wherein said inserter housing is releasably connected to said cannula trap.

12. The inserter of claim 10, wherein said cannula trap tilts by rotating about an axis that is parallel to a bottom surface of said base section and passes through said base section.

13. The inserter of claim 12, wherein at least one of said inserter housing and said base includes elements that bias said cannula trap to an upright position, such that upon completion of an insertion process with said cannula trap in a tilt position, said cannula trap is automatically returned to an upright position.

14. The inserter of claim 9, wherein said inserter housing is configured with a cut-out region that accommodates tilting of said inserter housing in relation to said base.

15. The inserter of claim 9, wherein at least one of said inserter housing and said base includes elements that bias said inserter housing to an upright position, such that upon completion of an insertion process with said inserter housing in a tilt position, said inserter housing is automatically returned to an upright position.
